(19) United States
(12) Patent Application Publication
(10) Pub. No.: US 2016/0278655 A1
(43) Pub. Date: Sep. 29, 2016

(54) ECG HIGH PASS FILTER

(71) Applicant: KONINKLIJKE PHILIPS N.V., Eindhoven (NL)

(72) Inventor: EARL CLARK HERLEIKSON, CINEBAR, WA (US)

(21) Appl. No.: 15/034,267

(22) PCT Filed: Nov. 5, 2014
(86) PCT No.: PCT/IB2014/065807

§ 371 Date: May 4, 2016

Related U.S. Application Data
(60) Provisional application No. 61/901,477, filed on Nov. 8, 2013.

(51) Int. Cl. A61B 5/04; A61N 1/39; A61B 5/0452; A61B 5/044; A61B 5/0408

(52) U.S. Cl. CPC A61B 5/04017; A61B 5/044; A61B 5/0408; A61B 5/0452; A61N 1/3925; A61N 1/3987

(57) ABSTRACT

An electrocardiogram high pass filter (25) employs a baseline low pass filter (40), a signal delay (43) and a signal extractor (44). In operations, baseline low pass filter (40) includes a finite impulse response low pass filter (41) and an infinite impulse response low pass filter (42) cooperatively low pass filtering a baseline unfiltered electrocardiogram signal (ECG_{bu}) to output a filtered baseline signal (BS_{ef}). Signal delay (43) time delays the baseline unfiltered electrocardiogram signal (ECG_{bu}) to output a delayed baseline unfiltered electrocardiogram signal (ECGdbu), and signal extractor (44) extracts the filtered baseline signal (BS_{ef}) from the delayed baseline unfiltered electrocardiogram signal (ECG_{dbu}) to output a baseline filtered electrocardiogram signal (ECG_{bf).}

The present invention generally relates to high pass filtering of electrocardiogram (“ECG”) signals. The present invention specifically relates to a high pass filtering of ECG signals for diagnostic and emergency medical service (“EMS”) purposes.

As known in the art, a signal amplitude of ECG signals is typically in the order of 1 mV, but may have a DC offset that varies from as much as −300 mV to +300 mV. This DC offset may drift with time and/or patient movement, and is often referred to as a “baseline wander”. Additionally, events such as defibrillation may have a dramatic effect on the baseline. In particular, a DC offset following a defibrillation event is usually drifting due to current that may flow through the ECG electrodes during the defibrillation event.

A typical ECG signal display setting for gain has a range of +/−2 mV in order to visually see a 1 mV ECG signal clearly. In response to potentially large and drifting DC offsets, high pass filters have been utilized to remove any DC offset in order to keep the ECG signal within view windows of a display and a printer. More particularly, a key diagnostic measurement of a ECG signal is the ST segment elevation or depression. This is performed by comparing a baseline of the ECG signal prior to a QRS with the baseline after the QRS. Ideally, the high pass filter should remove the baseline wander in such a way that the relative level of the baseline before and after the QRS is not affected.

ECG standards have been established that describe an impulse response requirement for diagnostic quality ECG measurements (e.g., EN 60601-2-27 and AAMI EC13). For example, an impulse applied in a standard test is 3 mV in amplitude with a duration of 100 mS, and the requirement is that a baseline should be displaced by less than 100 uV and a slope of the baseline should be less than 300 uV/sec following the impulse. Therefore, a high pass filter in an ECG system has conflicting goals.

Specifically, if the high pass filter is very responsive to the baseline wander in order to reliably maintain the baseline of the ECG signal in the center of the display, then it will also likely be responsive to the QRS such that the baseline following the QRS is displaced following the QRS by more than 100 uV. This is why an ECG monitor usually provides the clinician with several bandwidth settings for the high pass filter. The settings are often referred to as “Monitor” bandwidth for keeping the ECG signal visible on the display screen, and as “Diagnostic” bandwidth for making diagnostic ECG measurements (e.g., ST segment elevation and depression). Additionally, there is also the desire to display the ECG signal in real time with minimal time delay. This is important for clinical applications where timing is important such as synchronized cardioversion.

Historically, several types of high pass filters have been utilized in ECG monitors.

One such type of high pass filter for ECG monitors is an infinite impulse response (“IIR”) high pass filter that is computationally simple to implement. For example, a second order Butterworth high pass filter is easily implemented with five (5) multiply and accumulate calculations per sample with minimal time delay. However, a disadvantage of a IIR high pass filter is that a group delay is frequency dependent. This results in distortion of the ECG signal. Stated in another way, a IIR high pass filter responds to a positive ECG QRS signal by depressing the baseline following the ECG signal. Furthermore, in order to minimize the distortion to a level acceptable for diagnostic purposes, the corner frequency of the IIR high pass filter needs to be reduced to a frequency of 0.05 Hz or less. Additionally, a first order IIR high pass filter applied to a ramp will result in a DC offset and a second order IIR high pass filter applied to a ramp will result in a zero (0) DC offset. Thus, in order to remove a DC offset that is drifting following a defibrillation event, the IIR high pass filter would need to be at minimum a second order filter.

Another type of high pass filter for ECG monitors is a finite impulse response (“FIR”) high pass filter, which by definition has linear phase and constant group delay. Of note, a FIR high pass filter minimizes the distortion of the ECG signal due to the constant group delay and a 0.5 Hz or even a 0.67 Hz FIR high pass filter maybe implemented that meets the requirements for diagnostic quality ECG measurements in accordance ECG standards. Also, a FIR high pass filter responds well to a drifting DC offset following defibrillation, because it is usually designed to be symmetrical and an application of a FIR high pass filter to a ramp will produce a zero (0) DC offset. However, there are a couple of disadvantages of the FIR high pass filter. The first disadvantage is the time delay. Specifically, in order to have constant time delay for all frequencies, both the frequencies above and below the high pass corner frequency will see the same time delay, and a typical time delay is on the order of about one (1) second. The second disadvantage is the computational effort required. Specifically, a FIR high pass filter with one (1) second of time delay will have two (2) seconds of time history. A sample rate of 1000 Hz would require 2000 multiply accumulate calculations for each sample calculated at the 1000 Hz sample rate. Thus, for a full twelve (12) lead measurement, the number of multiply accumulate operations is 24M just for the FIR high pass filter.

Moreover, ECG monitoring is often performed on patients that are being moved. The out of hospital emergency medical services (“EMS”) typically see significant baseline wander of the ECG due to the movement of the patient. An EMS High pass filter is often provided for ECG systems designed for the EMS environment. This high pass filter will typically have a corner frequency in the range of 1 Hz to 2 Hz. A simple IIR filter with this high a corner frequency very substantially distorts the ECG waveform. A FIR filter with this corner frequency will minimize distortion of the ECG but would require a significant increase in computational effort.

To address the disadvantages of the prior art, the present invention provides an ECG high pass filter for diagnostic purposes (e.g., a corner frequency of 0.67 Hz or less) and EMS purposes (e.g., a corner frequency in the range of 1 Hz to 2 Hz). One form of the ECG high pass filter employs a baseline low pass filter, a signal delay and a signal extractor. In operation, the baseline low pass filter includes a finite impulse response low pass filter and an infinite impulse response low pass filter cooperatively low pass filtering a baseline unfiltered ECG signal to output a filtered baseline signal. The signal delay time delays the baseline unfiltered ECG signal to output a delayed baseline unfiltered ECG signal, and the signal extractor extracts the filtered baseline signal from the delayed baseline unfiltered ECG signal to output a baseline filtered ECG signal.

A second form of the present invention is a ECG monitor employing a processor to generate an ECG waveform of a heart of a patient and an ECG display to display the ECG waveform (e.g., visualized on a computer screen or in a printout). The processor incorporates the aforementioned ECG high pass filter of the present invention for diagnostic purposes and/or EMS purposes.

A third form of the present invention is a defibrillator, automatic or manual, employing an ECG monitor to generate an ECG waveform of a heart of a patient, a shock energy source to store shock energy and a defibrillation controller to control a delivery of the shock energy to the heart of the patient responsive to a QRS analysis of the electrocardiogram waveform. The ECG monitor incorporates the aforementioned ECG high pass filter of the present invention for diagnostic purposes and/or EMS purposes.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

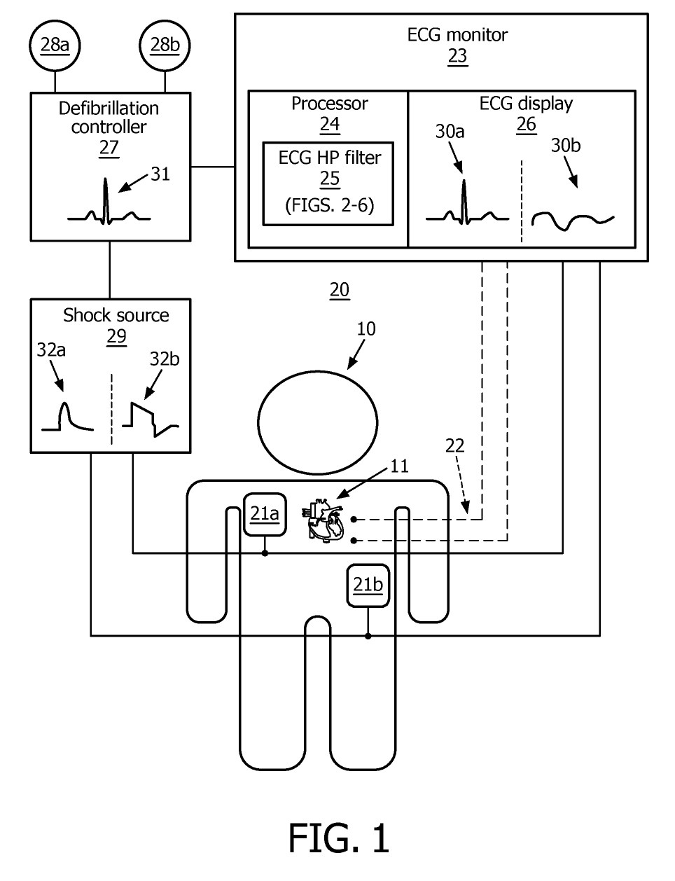

FIG. 1 illustrates an exemplary embodiment of a defibrillator with a ECG high pass filter in accordance with the present invention.

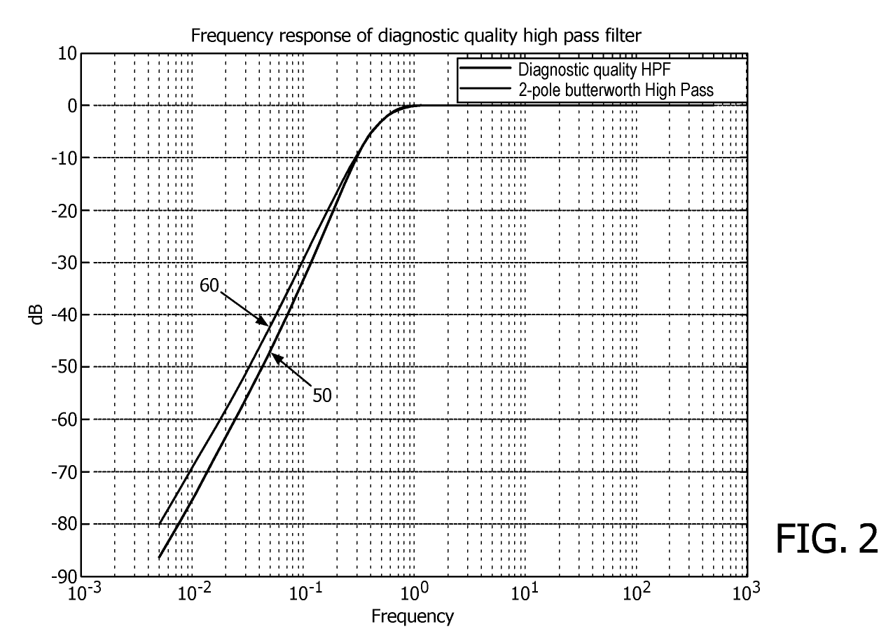

FIG. 2 illustrates exemplary frequency responses of a ECG high pass filter of the present invention and a 2-pole Butterworth high pass filter as known in the art.

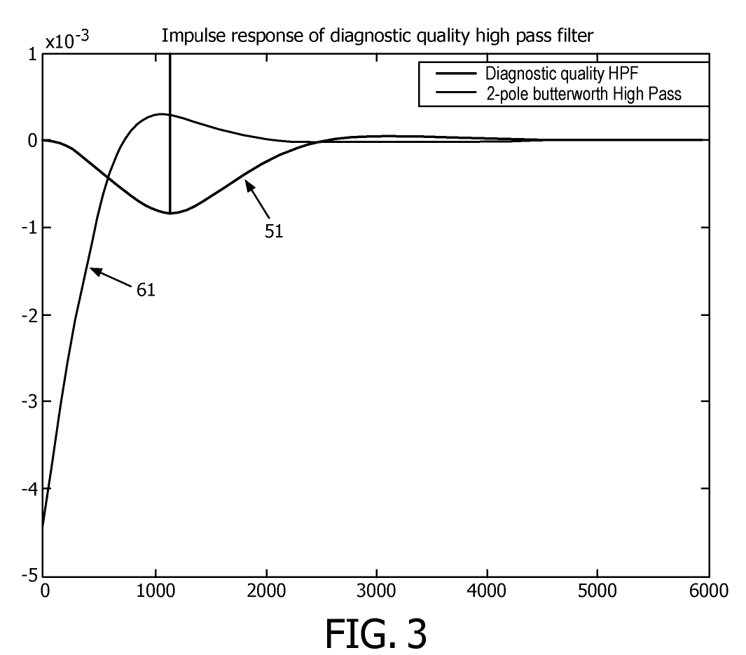

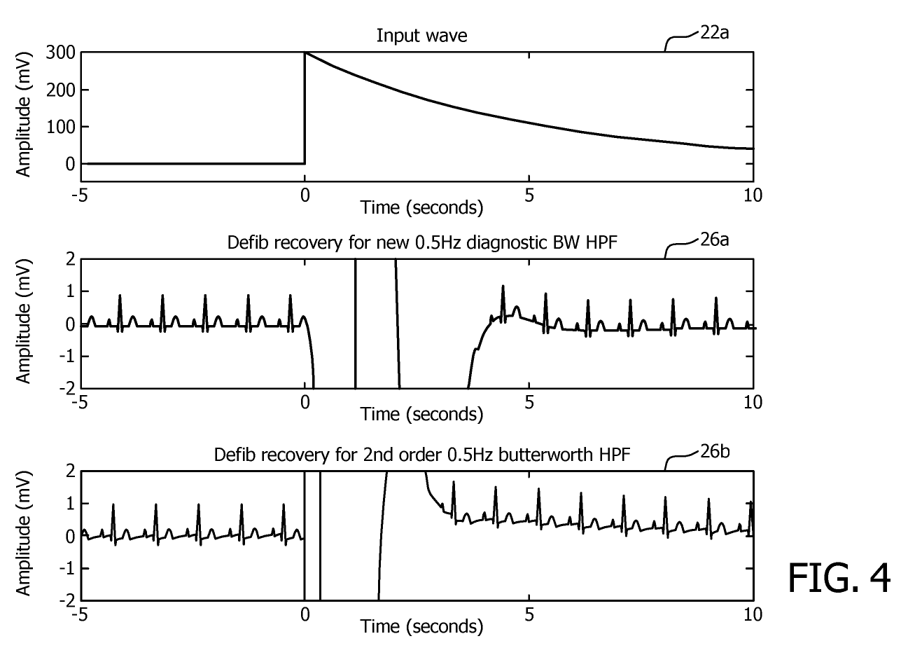

FIG. 3 illustrates exemplary impulse responses of a ECG high pass filter of the present invention and a 2-pole Butterworth high pass filter as known in the art. FIG. 4 illustrates exemplary defibrillation event recoveries of a ECG high pass filter of the present invention and a 2-pole Butterworth high pass filter as known in the art.

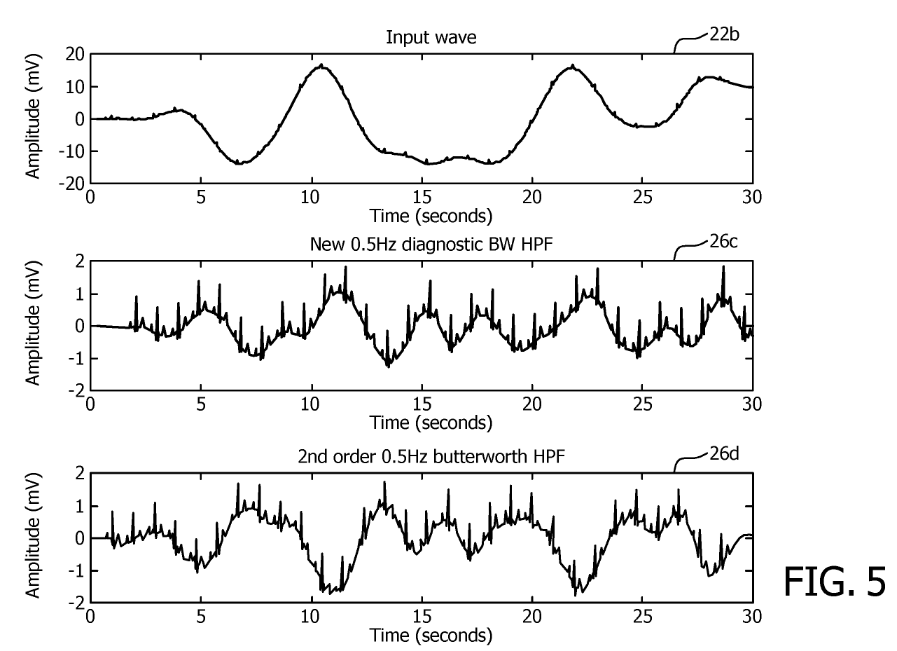

FIG. 5 illustrates exemplary baseline wander responses of a ECG high pass filter of the present invention and a 2-pole Butterworth high pass filter as known in the art.

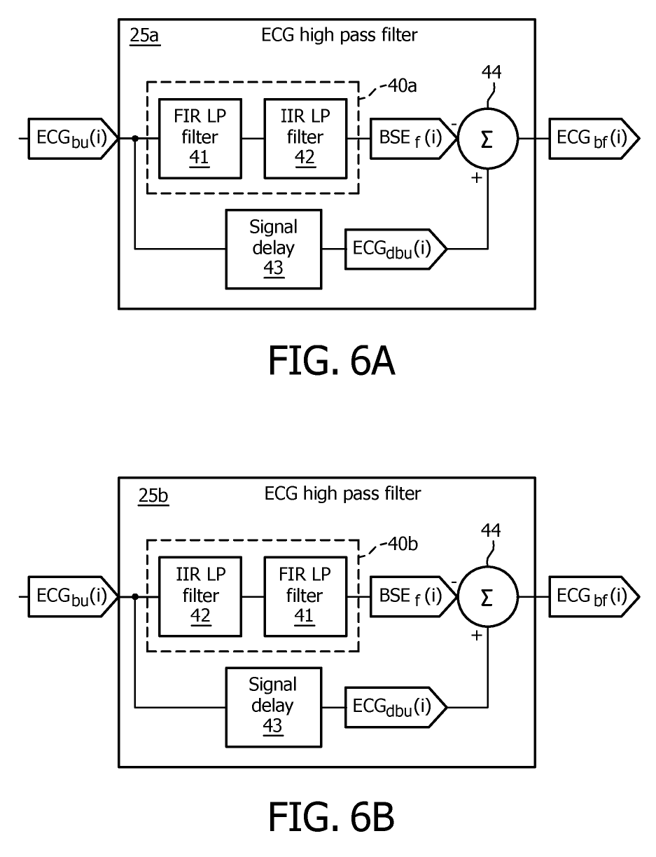

FIG. 6A illustrates a first exemplary embodiment of an ECG high pass filter in accordance with the present invention.

FIG. 6B illustrates a second exemplary embodiment of an ECG high pass filter in accordance with the present invention.

To facilitate an understanding of the present invention, exemplary embodiments of the present invention will be provided herein directed to ECG high pass filter for a defibrillator.

Referring to FIG. 1, a defibrillator 20 of the present invention employs a pair of electrode pads or paddles 21, optional ECG leads 22, a ECG monitor 23 (internal or external), a defibrillation controller 27. and a shock source 29.

Electrode pads/paddles 21 are structurally configured as known in the art to be conductively applied to a patient 10 in an anterior-apex arrangement as shown in FIG. 1 or in an anterior-posterior arrangement (not shown). Electrode pad/paddles 21 conduct a defibrillation shock from shock source 29 to a heart 11 of patient 10 and conduct an ECG signal (not shown) representative of electrical activity of heart 11 of patient 10 to ECG monitor 23. Alternatively or concurrently, ECG leads 22 are connected to patient 10 as known in the art to conduct the ECG signal to ECG monitor 23.

ECG monitor 23 is structurally configured as known in the art for processing the ECG signal to measure the electrical activity of heart 11 of patient 10 as an indication patient 10 is experiencing an organized heartbeat condition or an unorganized heartbeat condition. An example of the ECG signal indicating an organized heartbeat condition is an ECG waveform 30a that is representative of an organized contraction of the ventricles of heart 11 of patient 10 being capable of pumping blood. An example of the ECG signal indicating an unorganized heartbeat condition is an ECG waveform 30b that is representative of a ventricular fibrillation of heart 11 of patient 10.

To this end, ECG monitor 23 employs a processor 24 and a ECG display 26. For purposes of the present invention, processor 24 is broadly defined herein as any structurally arrangement of hardware, software, firmware and/or circuitry for executing functions required by ECG monitor 23 in processing the ECG signal. Generally, in operation, processor 24 is structurally configured to receive the ECG signal representative of the electrical activity of heart 11 of patient 10 in analog form from pads/paddles 21 and/or ECG leads 22, to condition as necessary and stream the ECG signal to defibrillation controller 27, and to generate the ECG waveform for display by ECG display 26. More particularly, in practice, processor 24 may implement analog-to-digital converters and various filters including a low pass filter having a corner frequency (e.g., ≧20 Hz) for filtering high frequency signals and a ECG high pass filter 25 of the present invention having a corner frequency (e.g., ≦2 Hz) for filtering low frequency signals like baseline wander/drift, particularly due to defibrillation events.

As will be explained further with the description herein of FIGS. 2-6, a structural design of ECG high pass filter 25 is a computationally simple design for execution by processor 24 that results in a minimal distortion of the ECG signal and in an excellent rejection of baseline wander/drift of the ECG signal.

For purposes of the present invention, ECG display 26 is broadly defined herein as any device structurally configured for presenting ECG waveform 30 for viewing including, but not limited to, a computer display and a printer.

Still referring to FIG. 1, shock source 29 is structurally configured as known in the art to store electric energy for delivery of a defibrillation shock 32 via electrode pads/paddles 21 to heart 11 of patient 10 as controlled by defibrillation controller 27. In practice, defibrillation shock 32 may have any waveform as known in the art. Examples of such waveforms include, but are not limited to, a monophasic sinusoidal waveform (positive sine wave) 32a and a biphasic truncated waveform 32b as shown in FIG. 1.

In one embodiment, shock source 29 employs a high voltage capacitor bank (not shown) for storing a high voltage via a high voltage charger and a power supply upon a pressing of a charge button 28a. Shock source 29 further employs a switching/isolation circuit (not shown) for selectively applying a specific waveform of an electric energy charge from the high voltage capacitor bank to electrode pads/paddles 21 as controlled by defibrillation controller 27.

Defibrillation controller 27 is structurally configured as known in the art to execute a manual synchronized cardioversion via a shock button 28b or an automatic synchronized cardioversion. In practice, defibrillation controller 27 employs hardware/circuitry (e.g., processor(s), memory, etc.) for executing a manual or an automatic synchronized cardioversion installed as software/firmware within defibrillation controller 27. In one embodiment, the software/firmware detects a QRS 31 of ECG signal 30 as a basis for controlling shock source 29 in delivering defibrillation shock 32 to heart 11 of patient 10.

Referring to FIGS. 2-6, a structural design of ECG high pass filter 25 in terms of operational performance and filter embodiments for achieving the operational performance will now be described herein to facilitate an understanding of the present invention.

Specifically, as to the operational performance for diagnostic purposes, FIGS. 2 and 3 respectively provide an exemplary frequency response and an exemplary impulse response of ECG high pass filter 25 as compared to a known 2-pole Butterworth monitor bandwidth high pass filter with each filter having a 3 db corner frequency of 0.5 Hz and a sample rate of input ECG signal of 1000 Hz. As shown in FIG. 2, a frequency response 50 of ECG high pass filter 25 has a better rejection performance of low frequency signals than a frequency response 60 of the known 2-pole Butterworth monitor bandwidth high pass filter. As shown in FIG. 3, an impulse response 51 of ECG high pass filter 25 has a substantially flat baseline of the inputted ECG signal prior to the impulse at the same level as the baseline after the impulse (i.e., an equivalent baseline before and after the impulse) while an impulse response 61 of the 2-pole Butterworth high pass filter has a very large baseline shift following the impulse.

Also by example, FIG. 4 shows an input wave 22a of the ECG signal having a defibrillation event at time Os with an offset change of 300 mV and an exponential decay of five (5) second time constant. For this example, a defibrillation recovery 26a of ECG high pass filter 25 has a similar performance to a defibrillation recovery 26b of the known 2-pole Butterworth monitor bandwidth high pass filter.

By further example, FIG. 5 shows a large level baseline wander 22b of the ECG signal. For this example, a center display 26c of the ECG signal as filtered by ECG high pass filter 25 has similar performance to a center display 26d of the ECG signal as filtered by the known 2-pole Butterworth monitor bandwidth high pass filter.

Referring to FIGS. 6A and 6B, structural embodiments of ECG high pass filter 25 for achieving such operational performance illustrated in FIGS. 2-5 include a baseline low pass filter 40 of the present invention, a signal delay 43 as known in the art and a signal extractor 44 as known in the art (e.g., an adder circuit). For embodiment 25a of ECG high pass filter 25, a baseline low pass filter 40a employs a series connection of FIR low pass filter 41 and a IIR low pass filter 42 as shown in FIG. 6A.

For embodiment 25b of ECG high pass filter 25, a baseline low pass filter 40b employs a series connection of IIR low pass filter 42 and FIR low pass filter 41 as shown in FIG. 6B.

For both embodiments, ECG high pass filter 25 is operated as an approximate linear phase filter having signal delay 43 for implementing as approximate linear phase filter response as applied to a baseline unfiltered electrocardiogram signal ECG_{bu}(i), which may have been previously low pass filtered for filtering high frequency signals (e.g., ≧20 Hz) and may have a predefined sample rate (e.g., a 1000 Hz). More importantly, baseline unfiltered electrocardiogram signal ECG_{bu}(i) may include a baseline wander/drift. In operation, baseline unfiltered electrocardiogram signal ECG_{bu}(i) is inputted into baseline low pass filter 40 and signal delay 43. A filtered baseline signal BSE_{f}(i) representative of any baseline wander/drift is outputted by baseline low pass filter 40 and extracted by signal extractor 44 from a delayed baseline unfiltered electrocardiogram signal ECG_{dbu}(i). The extraction yields a baseline filtered electrocardiogram signal ECG_{bf}(i) exhibiting minimal distortion and excellent rejection by baseline low pass filter 40 of any baseline wander/drift within baseline unfiltered electrocardiogram signal ECG_{bu}(i).

In practice, FIR low pass filter 41 and IIR low pass filter 42 are cooperatively structurally designed for low pass filtering baseline unfiltered electrocardiogram signal ECG_{b}(i) whereby baseline filtered electrocardiogram signal ECG_{bf}(i) is nonresponsive to a ramping of baseline unfiltered electrocardiogram signal ECG_{bu}(i) and/or a slope of the impulse response of baseline low pass filter 40 is substantially flat just prior to and after an impulse of baseline unfiltered electrocardiogram signal ECG_{bu}(i).

In one embodiment of FIR low pass filter 41, a boxcar FIR low pass filter is utilized where all of the coefficients are of the same value. As such, an implementation of the boxcar FIR low pass filter may be done by, at each sample interval, adding the input sample at the beginning of the boxcar FIR low pass filter and then subtracting it at the end of the boxcar FIR low pass filter.

An exemplary implementation of the boxcar FIR low pass filter for baseline low pass filter 40a (FIG. 6A) is in accordance with the following equation [1]:

$$w [ i ] = \frac{x [ i ] - x [ i - n ]}{n} + w [ i - 1 ] \qquad [ 1 ]$$

where w is the output of the boxcar FIR low pass filter 41, x is baseline unfiltered electrocardiogram signal ECG_{bu} is the number of coefficients in the boxcar FIR low pass filter.

An exemplary implementation of the boxcar FIR low pass filter for baseline low pass filter 40b (FIG. 6B) is in accordance with the following equation [2]:

$$w [ i ] = \frac{y [ i ] - y [ i - n ]}{n} + w [ i - 1 ] \qquad [ 2 ]$$

where w is filtered baseline signal BSE_{f,} y is the output of IIR low pass filter 42 and n is the number of coefficients in the boxcar FIR low pass filter.

In one embodiment of IIR low pass filter 42, a Butterworth 2^{nd} order low pass filter is utilized whereby the Butterworth 2^{nd} order low pass filter has a z-transform H(z) that may written in accordance with the following equation [3]:

$$H ( z ) = \frac{{b}_{0} + {b}_{1} {z}^{- 1} + {b}_{2} {z}^{- 2}}{1 + {a}_{1} {z}^{- 1} + {a}_{2} {z}^{- 2}} \qquad [ 3 ]$$

An exemplary implementation of the Butterworth 2^{nd} order low pass filter for baseline low pass filter 40a (FIG. 6A) is in accordance with the following equation [4]:

y[i]=b₀w[i]+b₁w[i−1]+b₂w[i−2]−a₁y[i−1]−a₂y[i−2] [4]

where y is filtered baseline signal BSE_{f}, w is the output of FIR low pass filter 41, and a and b are coefficients of the Butterworth 2^{nd} order low pass filter for setting a corner frequency of the Butterworth 2^{nd} order low pass filter.

An exemplary implementation of the Butterworth 2^{nd} order low pass filter for baseline low pass filter 40b (FIG. 6B) is in accordance with the following equation [5]:

y[i]=b₀x[i]+b₁x[i−1]+b₂x[i−2]−a₁y[i−1]−a₂y[i−2] [5]

where y is the output of the Butterworth 2^{nd} order low pass filter, x is baseline unfiltered electrocardiogram signal ECG_{bu}, and a and b are coefficients of the Butterworth 2^{nd} order low pass filter for setting a corner frequency of the Butterworth 2^{nd} order low pass filter.

One aspect of the cooperative structural configuration of FIR low pass filter 41 and IIR low pass filter 42 is a determination of a ratio of the number of coefficients n of the boxcar FIR low pass filter to the inverse of the corner frequency of the Butterworth 2^{nd} order low pass filter whereby baseline filtered electrocardiogram signal ECG_{bf}(i) is nonresponsive to a ramping of baseline unfiltered electrocardiogram signal ECG_{bu}(i). To this end, the corner frequency of the Butterworth 2^{nd} order low pass filter is computed as percentage of a desired corner frequency of ECG high pass filter 25, and the number of coefficients n of the boxcar FIR low pass filter is computed as a product of the inverse of the computed corner frequency of the Butterworth 2^{nd} order low pass filter normalized to one half the sample rate and a ratio as experimentally determined whereby baseline filtered electrocardiogram signal ECG_{bf}(i) is nonresponsive to a ramping of baseline unfiltered electrocardiogram signal ECG_{bu}(i). This aspect provides for optimal recovery of the ECG signal following a defibrillation event. This aspect also provides for the optimal rejection of low frequency baseline wander signals.

For an exemplary diagnostic implementation, the desired corner frequency of ECG high pass filter 25 is 0.5 Hz, the computed corner frequency of the Butterworth 2^{nd} order low pass filter is 72.2% of 0.5 Hz, and the number of coefficients n of the boxcar FIR low pass filter equals one-thousand-six (1006) samples for the length of the boxcar FIR low pass filter based on a ratio of 0.7267.

For an exemplary EMS implementation, the desired corner frequency of ECG high pass filter 25 is 1.917 Hz, the computed corner frequency of the Butterworth 2^{nd} order low pass filter is 72.5% of 1.917 Hz, and the number of coefficients n of the boxcar FIR low pass filter equals sixty-six (66) samples for the length of the boxcar FIR low pass filter based on a ratio of 0.7338.

A second aspect of the cooperative structural configuration of FIR low pass filter 41 and IIR low pass filter 42 is a gain of baseline low pass filter 40 being equal to a gain of signal delay 43. This aspect provides the optimal removal of the baseline wander signal.

A third aspect of the cooperative structural configuration of FIR low pass filter 41 and IIR low pass filter 42 is a time delay of a peak of an impulse response of the baseline low pass filter 40 being a basis for the time delaying of baseline unfiltered electrocardiogram signal ECG_{bu} the signal delay. This aspect provides optimal performance of the measurement of ST segment elevation or depression by minimizing the change of the baseline signal just prior to and just after the QRS wave.

Referring to FIGS. 1-6, those having ordinary skill in the art will appreciate numerous benefits of the present invention including, but not limited to, (1) substantial reduction in the computational requirement for implementing a ECG high pass filter that minimally distorts the ECG signal and has an excellent rejection of baseline wander/drift, particularly following a defibrillation event and (2) a ECG high pass filter configurable for both diagnostic purposes and EMS purposes.

While various embodiments of the present invention have been illustrated and described, it will be understood by those skilled in the art that the embodiments of the present invention as described herein are illustrative, and various changes and modifications may be made and equivalents may be substituted for elements thereof without departing from the true scope of the present invention. In addition, many modifications may be made to adapt the teachings of the present invention without departing from its central scope. Therefore, it is intended that the present invention not be limited to the particular embodiments disclosed as the best mode contemplated for carrying out the present invention, but that the present invention includes all embodiments falling within the scope of the appended claims.

1. An electrocardiogram high pass filter, comprising:
a baseline low pass filter including a finite impulse response low pass filter and an infinite impulse response low pass filter cooperatively structurally configured and operatively connected for low pass filtering a baseline unfiltered electrocardiogram signal (ECG_{bu}) to output a filtered baseline signal (BSE_{f});
a signal delay operable for time delaying the baseline unfiltered electrocardiogram signal (ECG_{bu}) to output a delayed baseline unfiltered electrocardiogram signal (ECG_{dbu}); and
a signal extractor operably connected to the baseline low pass filter and the signal delay for extracting the filtered baseline signal (BSE_{f}) from the delayed baseline unfiltered electrocardiogram signal (ECG_{dbu}) to output a baseline filtered electrocardiogram signal (ECG_{bf}).

2. The electrocardiogram high pass filter of claim 1, wherein the cooperative structural configuration of the finite impulse response low pass filter and the infinite impulse response low pass filter includes the baseline filtered electrocardiogram signal (ECG_{bf}) being nonresponsive to a ramping of the baseline unfiltered electrocardiogram signal (ECG_{bu}) as a ratio of a number of coefficients of the finite impulse response low pass filter to the inverse of a corner frequency of the infinite impulse response low pass filter.

3. The electrocardiogram high pass filter of claim 2, wherein the corner frequency of the infinite impulse response low pass filter is a function of a corner frequency of the electrocardiogram high pass filter.

4. The electrocardiogram high pass filter of claim 1, wherein the cooperative structural configuration of the finite impulse response low pass filter and the infinite impulse response low pass filter includes a gain of baseline low pass filter being equal to a gain of signal delay.

5. The electrocardiogram high pass filter of claim 1, wherein the cooperative structural configuration of the finite impulse response low pass filter and the infinite impulse response low pass filter includes a time delay of a peak of an impulse response of the baseline low pass filter being a basis for the time delaying of the baseline unfiltered electrocardiogram signal (ECG_{bu}) by the signal delay.

6. An electrocardiogram monitor, comprising:
a processor structurally configured to generate an electrocardiogram waveform of a heart of a patient, wherein the processor includes
a baseline low pass filter including a finite impulse response low pass filter and an infinite impulse response low pass filter cooperatively structurally configured and operatively connected for low pass filtering a baseline unfiltered electrocardiogram signal (ECG_{bu}) to output a filtered baseline signal (BSE_{f}),
a signal delay operable for time delaying the baseline unfiltered electrocardiogram signal (ECG_{bu}) to output a delayed baseline unfiltered electrocardiogram signal (ECG_{dbu}), and
a signal extractor operably connected to the baseline low pass filter and the signal delay for extracting the filtered baseline signal (BSE_{f}) from the delayed baseline unfiltered electrocardiogram signal (ECG_{dbu}) to output a baseline filtered electrocardiogram signal (ECG_{bf}); and
an electrocardiogram display structurally configured to display the electrocardiogram waveform.

7. The electrocardiogram monitor of claim 6, wherein the cooperative structural configuration of the finite impulse response low pass filter and the infinite impulse response low pass filter includes the baseline filtered electrocardiogram signal (ECG_{bf}) being nonresponsive to a ramping of the baseline unfiltered electrocardiogram signal (ECG_{bu}) as derived from a ratio of a number of coefficients of the finite impulse response low pass filter to the inverse of a corner frequency of the infinite impulse response low pass filter.

8. The electrocardiogram monitor of claim 7, wherein the corner frequency of the infinite impulse response low pass filter is a function of a corner frequency of the electrocardiogram high pass filter.

9. The electrocardiogram monitor of claim 6, wherein the cooperative structural configuration of the finite impulse response low pass filter and the infinite impulse response low pass filter includes a gain of baseline low pass filter being equal to a gain of signal delay.

10. The electrocardiogram monitor of claim 6, wherein the cooperative structural configuration of the finite impulse response low pass filter and the infinite impulse response low pass filter includes a time delay of a peak of an impulse response of the baseline low pass filter being a basis for the time delaying of the baseline unfiltered electrocardiogram signal (ECG_{bu}) by the signal delay.

11. A defibrillator comprising:
an electrocardiogram monitor structurally configured to generate an electrocardiogram waveform of a heart of a patient, wherein the electrocardiogram monitor includes
a baseline low pass filter including a finite impulse response low pass filter and an infinite impulse response low pass filter cooperatively structurally configured and operatively connected for low pass filtering a baseline unfiltered electrocardiogram signal (ECG_{bu}) to output a filtered baseline signal (BSE_{f}),
a signal delay operable for time delaying the baseline unfiltered electrocardiogram signal (ECG_{bu}) to output a delayed baseline unfiltered electrocardiogram signal (ECG_{dbu}), and
a signal extractor operably connected to the baseline low pass filter and the signal delay for extracting the filtered baseline signal (BSE) from the delayed baseline unfiltered electrocardiogram signal (ECG_{dbu}) to output a baseline filtered electrocardiogram signal (ECG_{bf});
a shock energy source structurally configured to store shock energy; and
a defibrillation controller structurally configured to control a delivery of the shock energy to the heart of the patient responsive to a QRS analysis of the electrocardiogram waveform.

12. The defibrillator of claim 11, wherein the cooperative structural configuration of the finite impulse response low pass filter and the infinite impulse response low pass filter includes the baseline filtered electrocardiogram signal (ECG_{bf}) being nonresponsive to a ramping of the baseline unfiltered electrocardiogram signal (ECG_{bu}) as derived from a ratio of a number of coefficients of the finite impulse response low pass filter to the inverse of a corner frequency of the infinite impulse response low pass filter.

13. The defibrillator of claim 12, wherein the corner frequency of the infinite impulse response low pass filter is a function of a corner frequency of the electrocardiogram high pass filter.

14. The defibrillator of claim 11, wherein the cooperative structural configuration of the finite impulse response low pass filter and the infinite impulse response low pass filter includes a gain of baseline low pass filter being equal to a gain of signal delay.

15. The defibrillator of claim 11, wherein the cooperative structural configuration of the finite impulse response low pass filter and the infinite impulse response low pass filter includes a time delay of a peak of an impulse response of the baseline low pass filter being a basis for the time delaying of the baseline unfiltered electrocardiogram signal (ECG_{bu}) by the signal delay.
